(19) United States
(12) Patent Application Publication
(10) Pub. No.: US 2003/0087286 A1
(43) Pub. Date: May 8, 2003

(54) Isolation of eukaryotic genomic DNA using magnetically responsive solid functionalized particles

(76) Inventor: Timothy A. Hodge, Cordova, TN (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 10/233,972

(22) Filed: Sep. 3, 2002

Related U.S. Application Data
(63) Continuation-in-part of application No. 09/945,952, filed on Sep. 4, 2001.
(60) Provisional application No. 60/230,371, filed on Sep. 6, 2000.

(51) Int. Cl. C12Q 1/68; C07H 21/04

(52) U.S. Cl. 435/6; 435/270; 536/25.4

(57) ABSTRACT

The present invention relates to a method to isolate fully methylated unamplified eukaryotic DNA made of hundreds-of-thousand to billions of base pairs from a lysate. The eukaryotic tissue samples are digested with protolytic enzymes in buffer releasing cellular contents. Magnetically responsive functionalized solid particles are added to the lysate. Polyethylene Glycol and a high salt concentration are added to the mixture to disrupt the hydrogen bonding in the lysate. The eukaryotic genomic DNA binds to the functionalized microparticles and is washed a number of time with an alcohol mixture to remove and denature proteins. The genomic DNA is the eluted off the magnetically responsive functionalized solid particles and used in downstream reactions.

### CROSS-REFERENCED TO RELATED APPLICATIONS

This application is a continuation-in-part of U.S. patent application Ser. No. 09/945,952 filed Sep. 4, 2001 which is a continuation-in-part of U.S. Provisional Patent Application Serial No. 60/230,371 filed Sep. 6, 2000. The entire disclosures of which are hereby incorporated by reference.

### BACKGROUND OF THE INVENTION

1. Field of the Invention

This invention relates to a method for isolation of eukaryotic genome DNA using magnetic particles.

2. Description of the Related Art

Hawkins in U.S. Pat. Nos. 5,898,071 and 5,705,628 teaches a method of separating polynucleotides, containing other polynucleotides by reversibly and nonspecifically binding the polynucleotides to a solid surface, such as magnetic microparticle, having a functional group-coated surface. The salt and polyalkylene glycol concentrations of the solution are adjusted to levels, which result in polynucleotide binding to the solid surface. The solid surface is separated from solution and the polynucleotides are separate from the magnetic microparticle. Hawkins teaches the use of this separation method for a plasmid, cosmid, single stranded DNA isolation from bacteriophages, PCR amplified DNA products and DNA fragments. Typically the aforementioned nucleic acids are hundreds or several thousand base pairs in length. Eukaryotic genomic DNA differs in that a fully intact genome has 3.0×10⁹ base pairs. Additionally, DNA repair mechanism utilize methylation which yeilds a highly methylated genome in eukaryotes.

Bitner et al., U.S. Pat. No.6,284,470 discloses a kit for cell concentration and lysate clearance using magnetic particles. Bitner et al. notes that the method can be used to isolate target nucleic acids including genomic DNA. The process disclosed by Bitner et al., however, differs from the present method in that the separation method is pH dependent “when the target nucleic acid is genomic DNA, it is necessary to disrupt the tissue to release the target genomic DNA from association with the other material in the tissue, so that target genomic DNA can adhere to the pH dependent ion exchange matrix in the presence of a solution at the first pH. The resulting complex and genomic DNA is separated from the disrupted tissue, and washed to remove additional contaminates (if necessary). The genomic DNA is then eluted from the complex by combining the complex with an elution solution having a second pH which is higher than the first pH.” '470 Patent at page 14. A simple process not dependent on pH or chaotropic salts would be beneficial.

### BRIEF SUMMARY OF THE INVENTION

The present invention relates to a method to isolate fully methylated unamplified eukaryotic DNA made of hundreds-of-thousand to billions of base pairs from a lysate. The eukaryotic tissue samples are digested with protolytic enzymes in buffer releasing cellular contents. Magnetically responsive functionalized solid particles are added to the lysate. Polyethylene Glycol and a high salt concentration are added to the mixture to disrupt the hydrogen bonding in the lysate. The eukaryotic genomic DNA binds to the magnetically responsive functionalized particles and is washed a number of time with an alcohol mixture to remove and denature proteins. The genomic DNA is the eluted off the magnetically responsive functionalized solid particles and used in downstream reactions.

More specifically, this invention related to a method to isolate genomic DNA involving the steps of: contacting biological lysate with magnetically responsive functionalized solid particles, adding a sufficient amount of a binding buffer to nonspecifically bind genomic DNA to the magnetically responsive functionalized solid particles to form bound genomic DNA, separating bound genomic DNA from the biological lysate, washing bound genomic DNA, eluting the bound genomic DNA and separating eluted genomic DNA from the magnetically responsive functionalized solid particles.

### BRIEF DESCRIPTION OF THE DRAWINGS

FIG. 1 is a bar graph showing bead volume versus average 260 nm measurement.

FIG. 2 is a bar graph showing bead iterations versus protein ratio.

FIG. 3 is a bar graph showing bead volume versus average 260 nm measurement.

FIG. 4 is a bar graph showing bead iteration versus protein ratio.

FIG. 5 is a bar graph showing PEG Percent averages versus average 260 nm measurement.

FIG. 6 is a bar graph showing PEG Percent ratios versus protein ratio.

FIG. 7 is a bar graph showing tissue 260 nm averages versus number of washes.

FIG. 8 is a bar graph showing tissue 260 nm/280 nm ratios versus number of washes.

FIG. 9 is a bar graph showing wash solutions versus 260 nm totals.

FIG. 10 is a bar graph showing wash solutions versus protein ratio.

FIG. 11 is a bar graph showing wash solutions versus 260 nm totals.

FIG. 12 is a bar graph showing wash solutions versus protein ratio.

FIG. 13 is a bar graph showing tissue mg versus average 260 nm measurement.

FIG. 14 is a bar graph showing tissue mg versus total 260 nm measurement.

### DETAILED DESCRIPTION OF THE PREFERRED EMBODIMENT

The present invention provides a method for isolating eukarotic genomic DNA. All patents, patent applications and articles discussed or referred to in this specification are hereby incorporated by reference.

The following terms and acronyms are used throughout the detailed description:

1. Definitions

complementary—chemical affinity between nitrogenous bases as a result of hydrogen bonding.

Responsible for the base pairing between nucleic acid strands. Klug, W. S. and Cummings, M. R. (1997) Concepts of Genetics, 5^{th} ed., Prentice-Hall, Upper Saddle River, N.J. (hereby incorporated by reference)

designated genetic sequence—includes a transgenic insert, a selectable marker, recombinant site or any gene or gene segment.

DNA (deoxyribonucleic acid)—The molecule that encodes genetic information. DNA is a double-stranded molecule held together by weak bonds between base pairs of nucleotides. The four nucleotides in DNA contain the bases: adenine (A), guanine (G) cytosine (C), and thymine (T). In nature, base pairs form only between A and T and between G and C; thus the base sequence of each single strand can be deduced from that of its partner.

genome—all the genetic material in the chromosomes of a particular organism; its size is generally given as its total number of base pairs.

genomic DNA—all of the genetic information encoded in a cell. Lehninger, A. L., Nelson, D. L. Cox, M. M. (1993) Principles of Biochemistry, 2^{nd} ed., Worth Publishers, New York, N.Y. (hereby incorporated by reference)

microarray technology—is a hybridization-based process that allows simultaneous quantitation of many nucleic acid species, has been described (M. Schena, D. Shalon, R. W. Davis, and P. O. Brown, “Quantititative Monitoring Of Gene Expression Patterns With A Complementary DNA Microarray,” Science, 270(5235), 467-70, 1995; J. DeRisi, L. Penland, P. O. Brown, M. L. Bittner, P. S. Meltzer, M. Ray, Y, Chen, Y. A. Su, and J. M. Trent, “Use Of A Cdna Microarray To Analyze Gene Expressions Patterns In Human Cancer,” Nature Genetics, 14(4), 457-60 (“DeRisi”), 1996; M. Schena, D. Shalon, R. Heller, A Chai, P. O. Brown, and R. W. Davis, “Parallel Human Genome Analysis: Microarray-Based Expression Monitoring Of 100 Genes,” Proc. Natl. Acad. Sci. USA., 93(20), 10614-9, 1996) hereby incorporated by reference., This technique combines robotic spotting of small amounts of individual, pure nucleic acids species on a glass surface, hybridization to this array with multiple fluorescently labeled nucleic acids, and detection and quantitation of the resulting fluor tagged hybrids with a scanning confocal microscope. This technology was developed for studying gene expression.

web site—a computer system that serves informational content over a network using the standard protocol of the World Wide Web. A Web Site corresponds to a particular Internet domain name such as TransnetYX.com.

Genomic DNA is isolated and purified using the separation method of magnetically responsive functionalized solid particles. The term “magnetically responsive” in the present specification means both magnetic and paramagnetic. The particles can range from 0.1 micron mean diameter to 100 microns in mean diameter. The particles can be functionalized as shown by Hawkins, U.S. Pat. No. 5,705,628 at col. 3 (hereinafter patent hereby incorporated by reference). More specifically, these nonsilica based particles have an iron oxide core with a solid phase surface functionalized with reactive groups such as a carboxyl group. In the preferred embodiment, the magnetic particles are 1 micron carboxylated iron core particles, but other magnetic particles with different functional groups of different size can be used.

For example a well of a wellplate is filled with biological lysate and magnetically responsive functionalized solid particles. The magnetically responsive functionalized solid particles can be dispensed into the well via a syringe pump or pipettor. A second syringe pump dispenses a binding buffer into the wells containing the raw biological material and active magnetically responsive functionalized solid particles. The dispensing itself may be sufficient to facilitate mixing of the samples with the particles. A secondary mixing mechanism, such as a tip can aspirated and re-dispense the liquid. A binding buffer, such as, 5%-20% polethylene glycol (PEG) 8000, 0.02% Sodium Azide and 2.5M Sodium Chloride is used to non-specifically bind the genomic DNA to the surface chemistry of the magnetically responsive functionalized solid particles. The PEG allows for hydrogen binding of water, which causes concentration of the DNA. The magnetically responsive functionalized solid particles, binding buffer and raw biological material are allowed to incubate at room temperature for ten minutes. After incubation, a magnet contacts the bottom of the well plate for several minutes, i.e. two to six minutes. The magnetic responsive functionalized solid particles with attached genomic DNA are magnetically attracted to the bottom of the master well plates forming a pellet of particles. The supernatant is removed. A wash buffer, for example 70% ethanol and 30% de-ionized water, is used to resuspend the magnetically responsive functionalized solid particles. The magnetically responsive functionalized solid particles, with the attached genomic DNA, are separated from the supernatant using a magnet. The supernatant is aspirated. The particle washing steps are repeated zero to five times.

The wellplates with pelletized particles are air-dried. In an alternative method, the pelletized particles can be dried with heat, compressed nitrogen or dried forced air. Once the particles are completely dry, the magnet is removed. The particles with attached genomic DNA are resuspended in a suspension buffer. A suspension buffer may be formulated to elute the bound DNA from the particles. In the preferred embodiment deionized water is used. Examples of formulated suspension buffers include 0.01 M Tris (pH 7.4), 0.02% Sodium Azide or Sodium Saline citrate (SSC), dimethyl sulfoxide (DMSO), sucrose (20%) or foramide (100%). In the preferred embodiment, the wellplates are heated to 80° C. for two minutes to disassociate the DNA from the particles.

After heating and resuspending the DNA in solution, the magnetic particles are separated from the purified DNA using a magnet. The supernatant is removed from the particles and pipetted into a secondary wellplate.

If a fully automated system is desired, the magnetic separator can be automated and rise from the bottom of the workstation and make contact with bottoms of all primary wellplates simultaneously.

In one embodiment, the genomic DNA can be sonicated before or after separation with the magnetically responsive functionalized solid particles. In the preferred embodiment, the genomic DNA is not sonicated after separation from the cellular debris. Sonication can be done by any conventional means such as a fixed horn instrument. Although sonication yields a wide range of fragments from about 100 base pairs to up to 1 kilobase, the average size of the fragment is around about 500 base pairs.

### EXAMPLE 1

Three to nine milligrams of mouse biopsy was added to a 96 wellplate. To each well containing biopsy 180 μl of Promega's (Madison, Wis.) Nuclei Lysis Solution with three milligrams of Proteinase K per milliliter was added. The plate was move to a 55° C. oven and allowed to incubate for one hour. The plate was vortexed five seconds. 136 μl of lysate was removed from each well and placed into a clean 384 deep wellplate. 55 μl of mixed carboxylated Seradyn (Indianapolis, Ind.) particles supplied via Agencourt (Beverly, Mass.) was added to each well containing lysate. 187 μl of 20% polyethylene glycol (PEG) 8000, 0.02% Sodium Azide and 2.5M Sodium Chloride was added to each sample. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated three more times. The particle pellets were allowed to dry in a 50° C. oven for 30 minutes. 30 μl of deionized water was added to each sample and allowed to incubate at room temperature for one minute. The samples were tip mixed eight times with a volume of 20 μl. The 384 deep wellplate was transferred back to the magnet for 1.5 minutes. 25 μl of eluate was transferred to a clean 96 UV optical wellplate. 5 μl of 20× Saline Sodium Citrate (SSC) was added to each sample in the optical plate. The samples were tip mixed three times with a volume of 25 μl. The optical plate was placed into an Optical Density reader (GENios; Serial number: 12900400173; Firmware: V 4.60-09/00 GENios; XFLUOR4 Version: V 4.20) and acquired 260 nm, 280 nm and 260/280 ratio reading. A typical reading from 12 samples is as follows:

TABLE 1

| <> | 1 | 2 | 3 | 4 | 5 | 6 | 7 | 8 | 9 | 10 |
|---|---|---|---|---|---|---|---|---|---|---|
| A | 0.4679 | . . . | . . . | . . . | . . . | . . . | . . . | . . . | . . . | . . . |
| B | 0.6729 | . . . | . . . | . . . | . . . | . . . | . . . | . . . | . . . | . . . |
| C | 0.4774 | . . . | . . . | . . . | . . . | . . . | . . . | . . . | . . . | . . . |
| D | 0.7939 | . . . | . . . | . . . | . . . | . . . | . . . | . . . | . . . | . . . |
| E | 0.3583 | . . . | . . . | . . . | . . . | . . . | . . . | . . . | . . . | . . . |
| F | 0.9081 | . . . | . . . | . . . | . . . | . . . | . . . | . . . | . . . | . . . |
| G | 0.4244 | . . . | . . . | . . . | . . . | . . . | . . . | . . . | . . . | . . . |
| H | 0.4975 | . . . | . . . | . . . | . . . | . . . | . . . | . . . | . . . | . . . |
| I | 0.5794 | . . . | . . . | . . . | . . . | . . . | . . . | . . . | . . . | . . . |
| J | 0.7966 | . . . | . . . | . . . | . . . | . . . | . . . | . . . | . . . | . . . |
| K | 0.4910 | . . . | . . . | . . . | . . . | . . . | . . . | . . . | . . . | . . . |
| L | 0.6325 | . . . | . . . | . . . | . . . | . . . | . . . | . . . | . . . | . . . |
| M | | . . . | . . . | . . . | . . . | . . . | . . . | . . . | . . . | . . . |
| N | | . . . | . . . | . . . | . . . | . . . | . . . | . . . | . . . | . . . |
| O | | . . . | . . . | . . . | . . . | . . . | . . . | . . . | . . . | . . . |
| P | | . . . | . . . | . . . | . . . | . . . | . . . | . . . | . . . | . . . |

Dual wave data (difference)

TABLE 2

| | | 2 | 3 | 4 | 5 | 6 | 7 | 8 | 9 | 10 |
|---|---|---|---|---|---|---|---|---|---|---|
| A | 0.6044 | . . . | . . . | . . . | . . . | . . . | . . . | . . . | . . . | . . . |
| B | 1.5218 | . . . | . . . | . . . | . . . | . . . | . . . | . . . | . . . | . . . |
| C | 0.6102 | . . . | . . . | . . . | . . . | . . . | . . . | . . . | . . . | . . . |
| D | 1.4098 | . . . | . . . | . . . | . . . | . . . | . . . | . . . | . . . | . . . |
| E | 0.4841 | . . . | . . . | . . . | . . . | . . . | . . . | . . . | . . . | . . . |
| F | 1.8873 | . . . | . . . | . . . | . . . | . . . | . . . | . . . | . . . | . . . |
| G | 0.6301 | . . . | . . . | . . . | . . . | . . . | . . . | . . . | . . . | . . . |
| H | 0.7039 | . . . | . . . | . . . | . . . | . . . | . . . | . . . | . . . | . . . |
| I | 0.6960 | . . . | . . . | . . . | . . . | . . . | . . . | . . . | . . . | . . . |
| J | 1.1770 | . . . | . . . | . . . | . . . | . . . | . . . | . . . | . . . | . . . |
| K | 0.6078 | . . . | . . . | . . . | . . . | . . . | . . . | . . . | . . . | . . . |
| L | 1.3739 | . . . | . . . | . . . | . . . | . . . | . . . | . . . | . . . | . . . |
| M | | . . . | . . . | . . . | . . . | . . . | . . . | . . . | . . . | . . . |
| N | | . . . | . . . | . . . | . . . | . . . | . . . | . . . | . . . | . . . |
| O | | . . . | . . . | . . . | . . . | . . . | . . . | . . . | . . . | . . . |
| P | | . . . | . . . | . . . | . . . | . . . | . . . | . . . | . . . | . . . |

Raw data (dual wave measurement with measurement filter)

TABLE 3

| | | 2 | 3 | 4 | 5 | 6 | 7 | 8 | 9 | 10 |
|---|---|---|---|---|---|---|---|---|---|---|
| A | 0.1365 | . . . | . . . | . . . | . . . | . . . | . . . | . . . | . . . | . . . |
| B | 0.8489 | . . . | . . . | . . . | . . . | . . . | . . . | . . . | . . . | . . . |
| C | 0.1328 | . . . | . . . | . . . | . . . | . . . | . . . | . . . | . . . | . . . |
| D | 0.6159 | . . . | . . . | . . . | . . . | . . . | . . . | . . . | . . . | . . . |
| E | 0.1258 | . . . | . . . | . . . | . . . | . . . | . . . | . . . | . . . | . . . |
| F | 0.9792 | . . . | . . . | . . . | . . . | . . . | . . . | . . . | . . . | . . . |
| G | 0.2057 | . . . | . . . | . . . | . . . | . . . | . . . | . . . | . . . | . . . |
| H | 0.2064 | . . . | . . . | . . . | . . . | . . . | . . . | . . . | . . . | . . . |
| I | 0.1166 | . . . | . . . | . . . | . . . | . . . | . . . | . . . | . . . | . . . |
| J | 0.3804 | . . . | . . . | . . . | . . . | . . . | . . . | . . . | . . . | . . . |
| K | 0.1168 | . . . | . . . | . . . | . . . | . . . | . . . | . . . | . . . | . . . |
| L | 0.7414 | . . . | . . . | . . . | . . . | . . . | . . . | . . . | . . . | . . . |
| M | | . . . | . . . | . . . | . . . | . . . | . . . | . . . | . . . | . . . |
| N | | . . . | . . . | . . . | . . . | . . . | . . . | . . . | . . . | . . . |
| O | | . . . | . . . | . . . | . . . | . . . | . . . | . . . | . . . | . . . |
| P | | . . . | . . . | . . . | . . . | . . . | . . . | . . . | . . . | . . . |

Raw data (dual wave measurement with reference filter)

TABLE 4

| <> | 1 | 2 | 3 | 4 | 5 | 6 | 7 | 8 | 9 | 10 |
|---|---|---|---|---|---|---|---|---|---|---|
| A | 0.2487 | . . . | . . . | . . . | . . . | . . . | . . . | . . . | . . . | . . . |
| B | 0.3632 | . . . | . . . | . . . | . . . | . . . | . . . | . . . | . . . | . . . |
| C | 0.2522 | . . . | . . . | . . . | . . . | . . . | . . . | . . . | . . . | . . . |
| D | 0.3982 | . . . | . . . | . . . | . . . | . . . | . . . | . . . | . . . | . . . |
| E | 0.1880 | . . . | . . . | . . . | . . . | . . . | . . . | . . . | . . . | . . . |
| F | 0.4814 | . . . | . . . | . . . | . . . | . . . | . . . | . . . | . . . | . . . |
| G | 0.2211 | . . . | . . . | . . . | . . . | . . . | . . . | . . . | . . . | . . . |
| H | 0.2999 | . . . | . . . | . . . | . . . | . . . | . . . | . . . | . . . | . . . |
| I | 0.3024 | . . . | . . . | . . . | . . . | . . . | . . . | . . . | . . . | . . . |
| J | 0.4799 | . . . | . . . | . . . | . . . | . . . | . . . | . . . | . . . | . . . |
| K | 0.2581 | . . . | . . . | . . . | . . . | . . . | . . . | . . . | . . . | . . . |
| L | 0.3920 | . . . | . . . | . . . | . . . | . . . | . . . | . . . | . . . | . . . |
| M | | . . . | . . . | . . . | . . . | . . . | . . . | . . . | . . . | . . . |
| N | | . . . | . . . | . . . | . . . | . . . | . . . | . . . | . . . | . . . |
| O | | . . . | . . . | . . . | . . . | . . . | . . . | . . . | . . . | . . . |
| P | | . . . | . . . | . . . | . . . | . . . | . . . | . . . | . . . | . . . |

Dual wave data (difference)

TABLE 5

| <> | 1 | 2 | 3 | 4 | 5 | 6 | 7 | 8 | 9 | 10 |
|---|---|---|---|---|---|---|---|---|---|---|
| A | 0.3855 | . . . | . . . | . . . | . . . | . . . | . . . | . . . | . . . | . . . |
| B | 0.6432 | . . . | . . . | . . . | . . . | . . . | . . . | . . . | . . . | . . . |
| C | 0.3860 | . . . | . . . | . . . | . . . | . . . | . . . | . . . | . . . | . . . |
| D | 0.9917 | . . . | . . . | . . . | . . . | . . . | . . . | . . . | . . . | . . . |
| E | 0.3143 | . . . | . . . | . . . | . . . | . . . | . . . | . . . | . . . | . . . |
| F | 1.4484 | . . . | . . . | . . . | . . . | . . . | . . . | . . . | . . . | . . . |
| G | 0.4238 | . . . | . . . | . . . | . . . | . . . | . . . | . . . | . . . | . . . |
| H | 0.4689 | . . . | . . . | . . . | . . . | . . . | . . . | . . . | . . . | . . . |
| I | 0.4187 | . . . | . . . | . . . | . . . | . . . | . . . | . . . | . . . | . . . |
| J | 0.8534 | . . . | . . . | . . . | . . . | . . . | . . . | . . . | . . . | . . . |
| K | 0.3765 | . . . | . . . | . . . | . . . | . . . | . . . | . . . | . . . | . . . |
| L | 1.0643 | . . . | . . . | . . . | . . . | . . . | . . . | . . . | . . . | . . . |
| M | | . . . | . . . | . . . | . . . | . . . | . . . | . . . | . . . | . . . |
| N | | . . . | . . . | . . . | . . . | . . . | . . . | . . . | . . . | . . . |
| O | | . . . | . . . | . . . | . . . | . . . | . . . | . . . | . . . | . . . |
| P | | . . . | . . . | . . . | . . . | . . . | . . . | . . . | . . . | . . . |

Raw data (dual wave measurement with measurement filter)

TABLE 6

| | | | | | | | | | | 10 |
|---|---|---|---|---|---|---|---|---|---|---|
| A | 0.1368 | . . . | . . . | . . . | . . . | . . . | . . . | . . . | . . . | . . . |
| B | 0.2800 | . . . | . . . | . . . | . . . | . . . | . . . | . . . | . . . | . . . |
| C | 0.1338 | . . . | . . . | . . . | . . . | . . . | . . . | . . . | . . . | . . . |
| D | 0.5935 | . . . | . . . | . . . | . . . | . . . | . . . | . . . | . . . | . . . |
| E | 0.1263 | . . . | . . . | . . . | . . . | . . . | . . . | . . . | . . . | . . . |
| F | 0.9670 | . . . | . . . | . . . | . . . | . . . | . . . | . . . | . . . | . . . |
| G | 0.2027 | . . . | . . . | . . . | . . . | . . . | . . . | . . . | . . . | . . . |
| H | 0.1690 | . . . | . . . | . . . | . . . | . . . | . . . | . . . | . . . | . . . |
| I | 0.1163 | . . . | . . . | . . . | . . . | . . . | . . . | . . . | . . . | . . . |
| J | 0.3735 | . . . | . . . | . . . | . . . | . . . | . . . | . . . | . . . | . . . |
| K | 0.1184 | . . . | . . . | . . . | . . . | . . . | . . . | . . . | . . . | . . . |
| L | 0.6723 | . . . | . . . | . . . | . . . | . . . | . . . | . . . | . . . | . . . |
| M | | . . . | . . . | . . . | . . . | . . . | . . . | . . . | . . . | . . . |
| N | | . . . | . . . | . . . | . . . | . . . | . . . | . . . | . . . | . . . |
| O | | . . . | . . . | . . . | . . . | . . . | . . . | . . . | . . . | . . . |
| P | | . . . | . . . | . . . | . . . | . . . | . . . | . . . | . . . | . . . |

Raw data (dual wave measurement with reference filter)

The most commonly used method of determining nucleic acid concentration is by performing an absorbance reading at 260 nm. Proteins have a tendency to absorb light at 280 nm. Table 2 represents the raw data reading for 260 nm and Table 5 represents the raw data reading for 280 nm. Since all substances, such as water and the optical plate, have some degree of a natural ability to absorb light, a reference wavelength should be used. Table 3 and Table 6 represents the data associated with a 999 nm reference wavelength reading. These values indicate the naturally occurring background noise. Table 1 (260 nm) represents the difference between Table 2 and Table 3. Table 4 (280 nm) represents the difference between Table 5 and Table 6. Subtracting the background noise from the raw yields a more accurate reading for both 260nm and 280 nm.

Table 7 represents the 260 nm/280 nm ratio. Nucleic acids absorb light at 260 nm and proteins absorb at 280 nm resulting in values that indicate the quantity of each substance. Dividing the DNA yield by the protein yield gives the DNA quality in terms of protein contamination. Stringent chemistries such a PCR and Sequencing are very intolerant of protein contamination.

TABLE 7

| 260 | 280 | Ratio |
|---|---|---|
| 0.4679 | 0.2487 | 1.881383 |
| 0.6729 | 0.3632 | 1.852698 |
| 0.4774 | 0.2522 | 1.892942 |
| 0.7939 | 0.3982 | 1.993722 |
| 0.3583 | 0.1880 | 1.905851 |
| 0.9081 | 0.4814 | 1.886373 |
| 0.4244 | 0.2211 | 1.919493 |
| 0.4975 | 0.2999 | 1.658886 |
| 0.5794 | 0.3024 | 1.916005 |
| 0.7966 | 0.4799 | 1.659929 |
| 0.4910 | 0.2581 | 1.902363 |
| 0.6325 | 0.3920 | 1.61352 |

### EXAMPLE 2

### Eukaryotic Genomic DNA Recovery and Purity with Varying Amounts of Magnetically Responsive Particles as Determined by Absorbance Measurements with a 0.25 cm Pathlength (30μl)

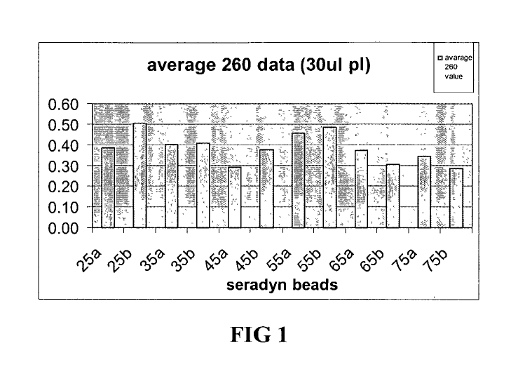

Ninety-six samples of 5 mg mouse tails were digested in 180 μl of Promega's (Madison, Wis.) Nuclei Lysis Solution (product number A7943) and 3 mg/ml Proteinase K. The samples were loaded with the lysis solution into a 96 wellplate and incubated at 600° C. for one hour. The wellplate was covered with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate Seradyn (Indianapolis, Ind.) carboxylated magnetically responsive particles supplied by Agencourt (Beverly, Mass.) were added. Six different bead volumes were each added to sixteen samples. The different bead volumes were 25 μl, 35 μl, 45 μl, 55 μl, 65 μl and 75 μl. To the bead lysate mixture was added. 187 μl of 20% polyethylene glycol (PEG) 8000, 0.02% sodium Azide and 2.5M Sodium Chloride. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated two more times. The particle pellets were allowed to air dry ten minutes. 50 μl of deionized water was added to each sample and allowed to incubate at room temperature for one minute. The samples were tip mixed eight times with a volume of 20 μl. The 384 deep wellplate was transferred back to the magnet for 1.5 minutes. 30 μl of DNA eluate was transferred to a clean 96 UV optical wellplate. The optical plate was placed into an Optical Density Instrument which acquired 260 nm, 280 nm and 260/280 ratio reading for a pathlength of 0.25 (30 μl), as shown in FIG. 1. FIG. 1 demonstrates, based on a 0.25 cm pathlength, that all the nucleic acid in the 5 mg sample was recovered with the minimum volume of beads. There is little appreciable difference in the average amounts of nucleic acid recovered with 25 μl of beads and 75 μl of beads.

The average 260 nm of eight samples for each bead volume is designated on the graph as the numerical volume of magnetically responsive particles in addition to the letter ‘a’ whereas the second eight samples of the same bead volume is designated on the graph as the numerical volume of magnetically responsive particles in addition to the letter ‘b’. The average 260 nm reading for each sample set was calculated and graphed.

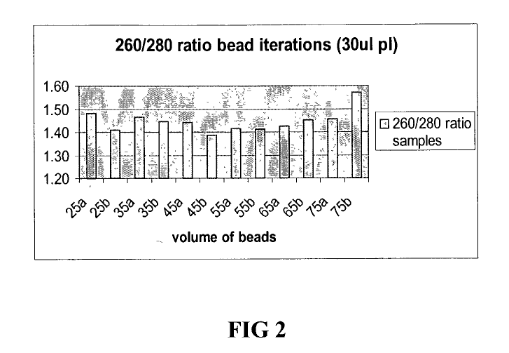

Additionally, each sample set had it 260 nm/280 nm ratio calculated as represent on the graph. The 260 nm/280 nm ratio shows the DNA to protein ratio, which indicated sample purity in terms of protein contamination, as shown in FIG. 2. FIG. 2 demonstrates that the DNA to protein (260 nm/280 nm) concentration are relatively consistent among each bead volume.

### EXAMPLE 3

### Eukaryotic Genomic DNA Recovery and Purity with Varying Amounts of Magnetically Responsive Particles as Determined by Absorbance Measurements with a 0.5 cm Pathlength (60μl)

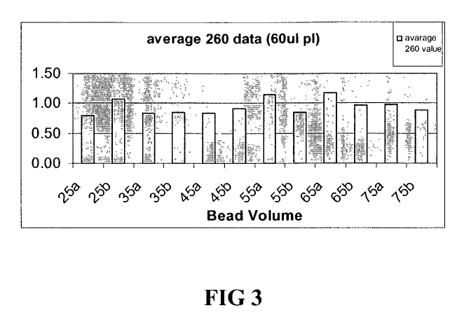

Ninety-six samples of 5 mg mouse tails were digested in 180 μl of Promega's (Madison, Wis.) Nuclei Lysis Solution (product number A7943) and 3 mg/ml Proteinase K. The samples were loaded with the lysis solution into a 96 wellplate and incubated at 60° C. for one hour. The wellplate was covered with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate Seradyn (Indianapolis, Ind.) carboxylated magnetically responsive particles supplied by Agencourt (Beverly, Mass.) were added. Six different bead volumes were each added to sixteen samples. The different bead volumes were 25 μl, 35 μl, 45 μl, 55 μl, 65μl and 75 μl. To the bead lysate mixture was added. 187 μl of 20% polyethylene glycol (PEG) 8000, 0.02% sodium Azide and 2.5M Sodium Chloride. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200μl of 70% ethanol wash solution was added to each well while still on the magnet. The particles were allowed to incubate three minutes. The 70% ethanol was removed and discarded. The wash process was repeated two more times. The particle pellets were allowed to air dry ten minutes. 75 μl of deionized water was added to each sample and allowed to incubate at room temperature for one minute. The samples were tip mixed eight times with a volume of 20μl. The 384 deep wellplate was transferred back to the magnet for 1.5 minutes. 60 μl of DNA eluate was transferred to a clean 96 UV optical wellplate. The optical plate was placed into an Optical Density Instrument which acquired 260 nm, 280 nm and 260/280 ratio reading for a pathlength of 0.5 cm (60 μl), as shown in FIG. 3. FIG. 3 values were determined with a 0.5 cm pathlength. Again as shown in FIG. 2, there does not appear to be any more nucleic acid recovered with an increased volume of beads. This suggests that both 25 μl-75 μl of bead is sufficient to recover all the nucleic acid from a 5 mg tissue sample.

The average 260 nm of eight samples for each bead volume is designated on the graph as the numerical volume of magnetically responsive particles in addition to the letter ‘a’ whereas the second eight samples of the same bead volume is designated on the graph as the numerical volume of magnetically responsive particles in addition to the letter ‘b’.

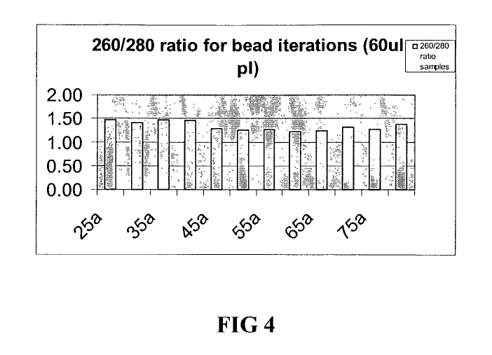

Additionally, each sample set had it 260 nm/280 nm ratio calculated as represent on the graph. The 260 nm/280 nm ratio shows the DNA to protein ratio, which indicated sample purity in terms of protein contamination, as shown in FIG. 4. FIG. 4 shows that the sample purity as determined by the 260 nm/280 nm are consistent among the different bead volumes.

### EXAMPLE 4

### Eukaryotic Genomic DNA Recovery and Purity with Varying Polyethylene Glycol as Determined by Absorbance Measurements with a 0.25 cm Pathlength (30 μl)

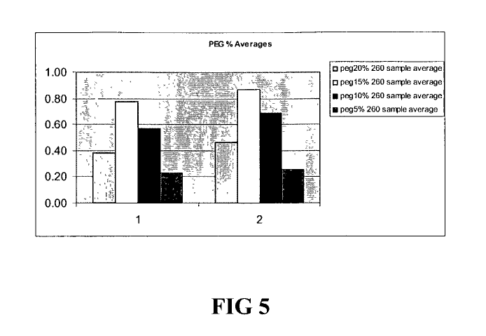

Sixty-four samples of 20 mg mouse tails was digested in 180 μl of Promega's (Madison, Wis.) Nuclei Lysis Solution (product number A7943) and 3 mg/ml Proteinase K. The samples were loaded with the lysis solution into a 96 wellplate and incubated at 60° C. for one hour. The wellplate was covered with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate 55 μl of Seradyn (Indianapolis, Ind.) carboxylated magnetically responsive particles supplied by Agencourt (Beverly, Mass.) were added. 187 μl of various percentages of polyethylene glycol (PEG) 8000, 0.02% Sodium Azide and 2.5M Sodium Chloride was added to the samples. 20%, 15%, 10% and 5% PEG was each added to sixteen samples. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated two more times. The particle pellets were allowed to air dry ten minutes. 50 μl of deionized water was added to each sample and allowed to incubate at room temperature for one minute. The samples were tip mixed eight times with a volume of 20 μl. The 384 deep wellplate was transferred back to the magnet for 1.5 minutes. 30 μl of DNA eluate was transferred to a clean 96 UV optical wellplate. The optical plate was placed into an Optical Density Instrument, which acquired 260 nm, 280 nm and 260/280 ratio reading for a pathlength of 0.25 cm (30 μl) as shown in FIG. 5. FIG. 5 shows that there is a marked increasing effect in the amount of nucleic acid recovered from samples treated with 5% PEG, 10% PEG and 15% PEG. However, there is a notable decrease in nucleic acid recovered with 20% PEG. Consequently, the preferred range of polyethylene glycol is between 5% to 15%, with the preferred amount of PEG at 15%

The average 260 nm for eight sample sets for each PEG percentage is designated on the graph as numeral ‘1’ whereas the second eight sample sets of the same PEG percentage is designated on the graph as the numeral ‘2’. For each sample set 20% PEG, 15% PEG, 10% PEG and 5% PEG is represented respectively.

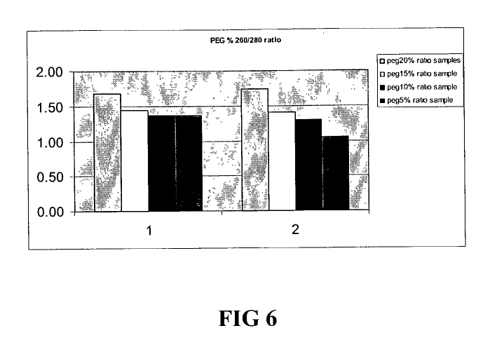

Additionally, each sample set had it 260 nm/280 nm ratio calculated as represent on the graph. The 260 nm/280 nm ratio shows the DNA to protein ratio, which indicated sample purity in terms of protein contamination, as shown in FIG. 6. FIG. 6 demonstrates a trend of increasing sample purity from 5%, 10%, 15% and 20% PEG. This suggests that the high concentration of PEG tends to denature proteins allowing for cleaner nucleic acid samples to be recovered.

### EXAMPLE 5

### Eukaryotic Genomic DNA Recovery and Purity with Varying Number of Ethanol Washes as Determined by Absorbance Measurements with a 0.25 cm Pathlength (30 μl)

Eighty samples of 20 mg mouse tails were digested in 180 μl of Promega's (Madison, Wis.) Nuclei Lysis Solution (product number A7943) and 3 mg/ml Proteinase K. The samples were loaded with the lysis solution into a 96 wellplate and incubated at 60° C. for one hour. The wellplate was covered with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate 55 μl of Seradyn (Indianapolis, Ind.) carboxylated magnetically responsive particles supplied by Agencourt (Beverly, Mass.) were added. 187 μl of 20% polyethylene glycol (PEG) 8000, 0.02% sodium Azide and 2.5M Sodium Chloride was added to the samples. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated zero to four more times. The particle pellets were allowed to air dry ten minutes. 50 μl of deionized water was added to each sample and allowed to incubate at room temperature for one minute. The samples were tip mixed eight times with a volume of 20 μl. The 384 deep wellplate was transferred back to the magnet for 1.5 minutes. 30 μl of DNA eluate was transferred to a clean 96 UV optical wellplate. The optical plate was placed into an Optical Density Instrument which acquired 260 nm, 280 nm and 260/280 ratio reading for a pathlength of 0.25 cm (30 μl), as shown in FIG. 7. FIG. 7 shows that the number of ethanol washes sample receive has little to no effect on the amount of nucleic acid recovered. On the graph the left set of samples shows a relative consistent sample recovery. The right set of samples on the graph suggest there may be some sample loss of nucleic acid during wash. The minimal sample loss may be due to some nucleic acid becoming unbound from the beads during multiple washes.

The average 260 nm for eight samples for each set of washes is designated on the graph in the left grouping whereas the second eight samples for each set of washes is designated on the graph in the right grouping. The left and right groupings represent one washing, two washing, three washings, four washings and five washing respectively.

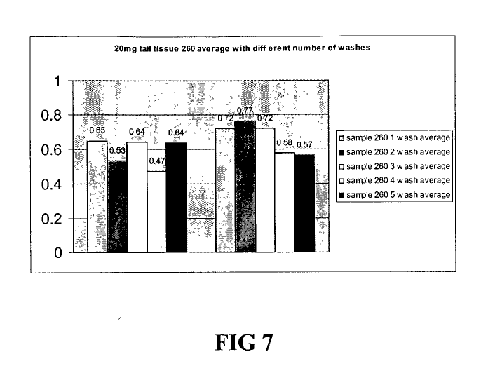

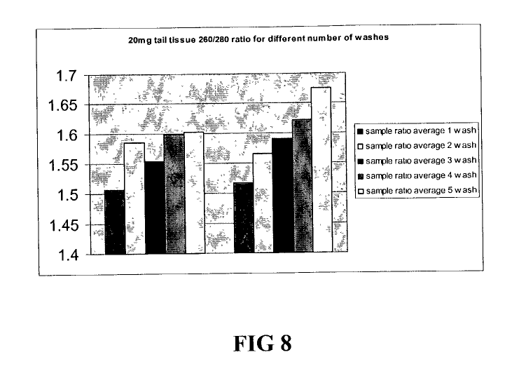

Additionally, each sample set had it 260 nm/280 nm ratio calculated as represent on the graph. The 260 nm/280 nm ratio shows the DNA to protein ratio, which indicated sample purity in terms of protein contamination, as shown in FIG. 8. FIG. 8 demonstrates that the number of ethanol washes has a profound effect on sample purity. The sample purity increased from one wash, two washes, three washes, four washes to five washes. It appears the more washes lead to a greater chance for proteins to soluabilize into the wash buffer.

### EXAMPLE 6

### Eukaryotic Genomic DNA Recovery and Purity with Varying Elution Solution and Volumes as Determined by Absorbance Measurements with a 0.25 cm Pathlength (30 μl)

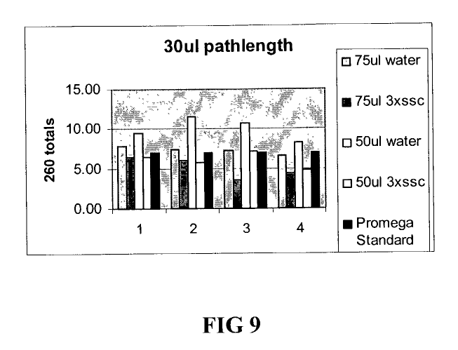

One hundred and twenty eight samples of 20 mg mouse tails were digested in 180 μl of Promega's (Madison, Wis.) Nuclei Lysis Solution (product number A7943) and 3 mg/ml Proteinase K. The samples were loaded with the lysis solution into a 96 wellplate and incubated at 60° C. for one hour. The wellplate was covered with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate 55 μl of Seradyn (Indianapolis, Ind.) carboxylated magnetically responsive particles supplied by Agencourt (Beverly, Mass.) were added. 187 μl of 20% polyethylene glycol (PEG) 8000, 0.02% Sodium Azide and 2.5M Sodium Chloride was added to the samples. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated two more times. The particle pellets were allowed to air dry ten minutes. The elution solutions and volumes were varied. 75 μl of water, 75 μl of 3× Saline Sodium Citrate (SSC), 50 μl of water, 50 μl of 3×SSC was added to sixteen samples and allowed to incubate at room temperature for one minute. The samples were tip mixed eight times with a volume of 20 μl. The 384 deep wellplate was transferred back to the magnet for 1.5 minutes. 30 μl of DNA eluate was transferred to a clean 96 UV optical wellplate. The optical plate was placed into an Optical Density Instrument, which acquired 260 nm, 280 nm and 260/280 ratio reading for a pathlength of 0.25 cm (30 μl), as shown in FIG. 9. FIG. 9 indicates that nucleic acid can be eluted in both water and 3×SSC. The graph indicates that more DNA is consistently recovered in the two different volumes of water than in either volume of 3×SSC.

The 260 nm sum for eight samples for each elution series is represented on the graph in four groupings. 75 μl of water, 75 μl of 3×SSC, 50 μl of water, 50 μl of 3×SSC and a Promega (Madison, Wis.) standard DNA are represented in each of the four grouping respectively. The Promega (Madison, Wis.) standard DNA had a concentration of 190 ng/μl. The standard DNA was not isolated in the process it was simply added to the optical plate to serve as a reference

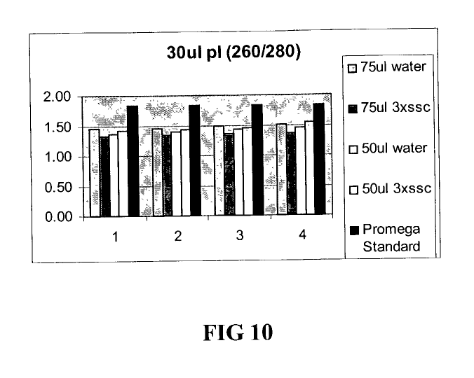

Additionally, each sample set had it 260 nm/280 nm ratio calculated as represent on the graph. The 260 nm/280 nm ratio shows the DNA to protein ratio, which indicated sample purity in terms of protein contamination, as shown in FIG. 10. FIG. 10 demonstrates regardless if the samples are eluted in water or 3×SSC at either volume, there is little if any significant impact on purity of the nucleic acid recovered. This is shown on the graph by the 260 nm/280 nm ratio is a 0.25 cm pathlength.

### EXAMPLE 7

### Eukaryotic Genomic DNA Recovery and Purity with Varying Elution Solution and Volumes as Determined by Absorbance Measurements with a 0.5 cm Pathlength (60 μl)

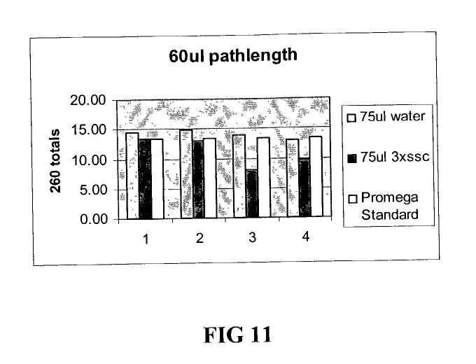

Sixty-four samples of 20 mg mouse tails were digested in 180 μl of Promega's (Madison, Wis.) Nuclei Lysis Solution (product number A7943) and 3 mg/ml Proteinase K. The samples were loaded with the lysis solution into a 96 wellplate and incubated at 600° C. for one hour. The wellplate was covered with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate 55 μl of Seradyn (Indianapolis, Ind.) carboxylated magnetically responsive particles supplied by Agencourt (Beverly, Mass.) were added. 187 μl of 20% polyethylene glycol (PEG) 8000, 0.02% sodium Azide and 2.5M Sodium Chloride was added to the samples. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated two more times. The particle pellets were allowed to air dry ten minutes. The elution solutions were varied. Thirty-two samples were eluted with 75 μl of water and thirty-two samples were eluted with 75 μl 3×SSC. The samples were allowed to incubate at room temperature for one minute. The samples were tip mixed eight times with a volume of 20 μl. The 384 deep wellplate was transferred back to the magnet for 1.5 minutes. 60 μl of DNA eluate was transferred to a clean 96 UV optical wellplate. The optical plate was placed into an Optical Density Instrument, which acquired 260 nm, 280 nm and 260/280 ratio reading for a pathlength of 0.5 cm (60 μl), as shown in FIG. 11. FIG. 11 again demonstrates that nucleic acid is recoverable in both water and 3×SSC as measures with a 0.5 cm pathlength. Water seems to be a solution that consistently is able to recover more DNA than 3×SSC.

The 260 nm sum for eight samples for each elution series is represented on the graph in four groupings. 75 μl of water, 75 μl of 3×SSC and a Promega (Madison, Wis.) standard DNA are represented in each of the four grouping respectively. The Promega (Madison, Wis.) standard DNA had a concentration of 190 ng/μl. The standard DNA was not isolated in the process it was simply added to the optical plate to serve as a reference.

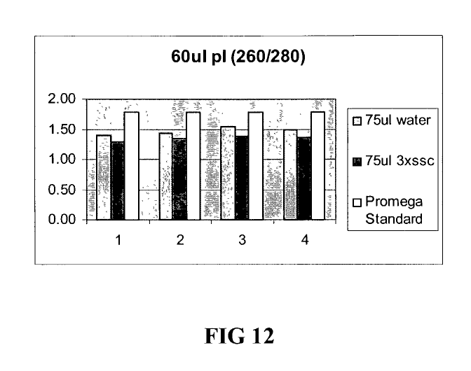

Additionally, each sample set had it 260 nm/280 nm ratio calculated as represent on the graph. The 260 nm/280 nm ratio shows the DNA to protein ratio, which indicated sample purity in terms of protein contamination, as shown in FIG. 12. FIG. 12 shows that there is a slight trend for the purity of the nucleic acid recovered in 3×SSC to be less than the purity of samples recovered in water. This was determined by absorbance measurements with a pathlength of 0.5 cm from the 260 nm/280 nm.

### EXAMPLE 8

### Eukaryotic Genomic DNA Recovery and Purity with Varying Amounts of Mouse Tail Tissue as Determined by Absorbance Measurements with a 0.5 cm Pathlength (60 μl)

Eight samples of 5 mg, eight samples of 10 mg, eight samples of 15 mg and eight samples of 20 mg mouse tail tissue was digested in 180 μl of Promega's (Madison, Wis.) Nuclei Lysis Solution (product number A7943) and 3 mg/ml Proteinase K. The samples were loaded with the lysis solution into a 96 wellplate and incubated at 60° C. for one hour. The wellplate was covered to with sealing tape to prevent evaporation. The digested tails were vortexed for five seconds. All the following steps were conducted with a Tecan (Durham, N.C.) Genesis liquid handler. 136 μl of lysate was removed and placed into a clean 400 μl 384 deep wellplate. To each sample of lysate 55 μl of Seradyn (Indianapolis, Ind.) carboxylated magnetically responsive solid particles supplied by Agencourt (Beverly, Mass.) were added. 187 μl of 20% polyethylene glycol (PEG) 8000, 0.02% sodium Azide and 2.5M Sodium Chloride. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated two more times. The particle pellets were allowed to air dry ten minutes. 75 μl of deionized water was added to each sample and allowed to incubate at room temperature for one minute. The samples were tip mixed eight times with a volume of 20 μl. The 384 deep wellplate was transferred back to the magnet for 1.5 minutes. 60 μl of DNA eluate was transferred to a clean 96 UV optical wellplate. The optical plate was placed into an Optical Density Instrument, which acquired 260 nm for a pathlength of 0.5 cm (60 μl), as shown in FIG. 13. FIG. 13 indicates that at a consistent volume of beads, more average nucleic acid is recovered from larger tissue samples. The trend increases from 5 mg, 10 mg, 15 mg and 20 mg of tissue samples. This may suggest that all the beads binding capacity is not utilized in smaller sample amounts. Consequently, at least 20 mg of samples can be used with one micron beads.

The average of eight samples for each tissue amount is designated on the graph. From left to right 5 mg, 10 mg, 15 mg and 20 mg are represented respectively.

Additionally, each sample set had it 260 nm sum calculated and represent on the graph. From left to right 5 mg, 10 mg, 15 mg and 20 mg are represented respectively, as shown in FIG. 14. FIG. 14 demonstrates a correlation with FIG. 13. FIG. 14 represents the sum of the 260 nm values. There is a consistent upward trend in the total amount of nucleic acid recovered from 5 mg, 10 mg, 15 mg and 20 mg as determined by the 260 nm measurement.

Although the present invention has been described and illustrated with respect to a preferred embodiment and a preferred use thereof, it is not to be so limited since modifications and changes can be made therein which are fully within the scope of the invention.

I claim:

1. A method to isolate genomic DNA comprising: contacting biological lysate with magnetically responsive functionalized solid particles, adding a sufficient amount of a binding buffer to nonspecifically bind genomic DNA to said magnetically responsive functionalized solid particles to form bound genomic DNA, separating said bound genomic DNA from said biological lysate, washing said bound genomic DNA, eluting said bound genomic DNA and separating eluted genomic DNA from said magnetically responsive functionalized solid particles.

2. The method of claim 1 wherein said magnetically responsive functionalized solid particles are functionalized with carboxyl groups.

3. The method of claim 2 wherein said magnetically responsive functionalized solid particles are one micron in diameter.

4. The method of claim 1 wherein the bound genomic DNA is eluted with water.

5. The method of claim 1 wherein said bound genomic DNA is washed at least five times.

6. The method of claim 1 wherein the binding buffer is polyethylene glycol.

7. The method of claim 6 wherein the preferred range of polyethylene glycol is between 5% to 15%.

8. The method of claim 6 wherein the amount of polyethylene glycol is 15%.

9. The method of claim 1 wherein said biological lysate includes between 5 to 20 mg of tissue samples.

10. The method of claim 1 wherein said biological lysate includes at least 20 mg of tissue samples.

11. The method of claim 1 wherein said genomic DNA is eukaryotic.
